(19) United States
(12) Patent Application Publication
(10) Pub. No.: US 2025/0122137 A1
(43) Pub. Date: Apr. 17, 2025

(54) METHOD FOR PREPARING 1,2-PENTANEDIOL

(71) Applicant: ZHEJIANG BOJU NEW MATERIALS CO., LTD., Zhejiang (CN)

(72) Inventors: Minghe ZHOU, Zhejiang (CN); Jun ZHOU, Zhejiang (CN); Zhaochang ZHOU, Zhejiang (CN)

(21) Appl. No.: 18/813,947

(22) Filed: Aug. 23, 2024

(30) Foreign Application Priority Data
Oct. 13, 2023 (CN) 202311323338.6

(51) Int. Cl. C07C 29/157; C07C 29/74

(52) U.S. Cl. CPC C07C 29/157; C07C 29/74; C07C 2523/755

(57) ABSTRACT

Provided is a method for preparing 1,2-pentanediol, including subjecting 2-hydroxypentanal and hydrogen to hydrogenation reduction under an action of a catalyst to obtain the 1,2-pentanediol; wherein the catalyst is a supported nickel-based catalyst; the supported nickel-based catalyst comprises a carrier and an active component supported on the carrier; the active component comprises a first component and a second component; the first component is a nickel compound; and the second component is one or more selected from the group consisting of a copper compound, a cobalt compound, a platinum compound, an iridium compound, a rhodium compound, and a rhenium compound.

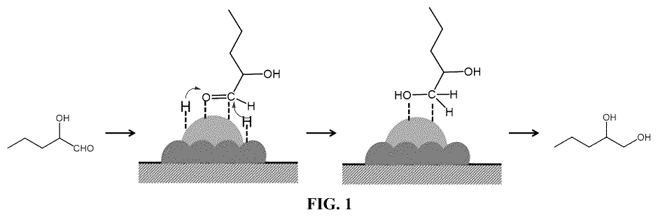

### CROSS REFERENCE TO RELATED APPLICATION

This patent application claims the benefit of and priority to Chinese Patent Application No. 202311323338.6 filed with the China National Intellectual Property Administration on Oct. 13, 2023, and entitled “METHOD FOR PREPARING 1,2-PENTANEDIOL”, the disclosure of which is incorporated by reference herein in its entirety as part of the present application.

### TECHNICAL FIELD

The present disclosure relates to the technical field of organic synthesis, and in particular to a method for preparing 1,2-pentanediol.

### BACKGROUND

1,2-pentanediol is a linear diol with distinct polar and non-polar properties, making it have different properties from other diols. 1,2-pentanediol is widely used throughout the world, mainly as an intermediate for the fungicide propiconazole and as a “no-additive” preservative for cosmetics, and can also be used to produce polyester fibers, surfactants, pharmaceuticals and other products.

There are two existing methods for producing 1,2-pentanediol in China:

The first method is to prepare 1,2-pentanediol by oxidating 1-pentene to obtain 1,2-epoxypentane, and then subjecting 1,2-epoxypentane to hydrolysis. For example, Chinese patent publication No. CN1552684A discloses a method for preparing 1,2-pentanediol using n-pentene, formic acid, and hydrogen peroxide as raw materials, including: (1) adding the formic acid and the hydrogen peroxide to a reactor at a low temperature; (2) adding a solution of n-pentene, and then conducting heat preservation for a period of time to allow a reaction to be sufficient; (3) conducting hydrolysis under an alkaline condition to obtain crude 1,2-pentanediol; (4) extracting the crude 1,2-pentanediol with an organic solvent; and (5) distilling and recovering the solvent from a resulting organic phase, and rectifying to obtain high purity 1,2-pentanediol. Chinese patent publication No. CN101885668A discloses a novel method for preparing 1,2-pentanediol using chloropentanol as a main raw material, including the following steps: adding the chloropentanol and an aqueous solution of an alkali to a reactor at room temperature; stirring, heating to a temperature of 60° C. to 80° C. and holding the temperature for a period of time to allow a full reaction; subjecting a resulting reaction product to neutralization with an acid or atmospheric distillation, and layering to obtain 1,2-epoxypentane; adding an organic solvent, a hydration catalyst, and water to the 1,2-epoxypentane, stirring, heating to a temperature of 30° C. to 100° C. and holding the temperature for a period of time to allow a full reaction; and layering a resulting reaction product, neutralizing a resulting aqueous layer with an alkali when it is acidic, dehydrating under reduced pressure to obtain crude 1,2-pentanediol, and subjecting the crude 1,2-pentanediol to rectification to obtain high-purity 1,2-pentanediol. Chinese patent publication No. CN104926600A discloses a method for synthesizing 1,2-pentanediol with a high yield by a one-pot process using n-pentene as a raw material, In the method, the reaction temperature is low, the risk of leakage caused by high-temperature volatilization of the raw material 1-pentene is reduced, and the pressure resistance requirement of equipment is reduced. In addition, the multi-step one-pot process is adopted to shorten the process flow. ZHENG Yaqing et al. (ZHENG Yaqing, DING Kehong, YANG Jin et al. Synthesis of propiconazole intermediate 1,2-pentanediol [J]. Pesticides, 2014.53 (2): 96-98) discloses that n-pentene, chlorine, and water are used as main raw materials to synthesize 1,2-pentanediol through addition, saponification, and hydrolysis, and some of the main influencing factors of a 2-step reaction of saponification and hydrolysis are investigated.

The second method is to conduct first selective hydrogenation on tetrahydrofurfural to obtain tetrahydrofurfuryl alcohol, which is then subjected to second selective hydrogenation to obtain the 1,2-pentanediol. For example: Chinese patent publication No. CN102924232A discloses that furfural is used as a raw material, a composite oxide containing copper oxide is used as a catalyst, and 1,2-pentanediol is prepared by hydrogenation in a continuous fixed bed by catalysis; wherein the hydrogenation is conducted at a temperature of 100° C. to 200° C. at a pressure of 4.0 MPa to 10.0 MPa. The conversion of furfural and the selectivity of 1,2-pentanediol are high. The renewable resource furfural is used for the production of 1,2-pentanediol, which has abundant raw material sources and low cost. Chinese patent publication No. CN104370702A discloses a method for preparing 1,2-pentanediol by liquid-phase selective hydrogenolysis of furfuryl alcohol. A green and efficient non-precious metal catalyst, specifically a supported Cu catalyst, is selected to prepare the 1,2-pentanediol with high activity and selectivity under mild hydrogenation conditions with furfural. The non-precious metal catalyst can achieve high-concentration or even pure furfuryl alcohol as a raw material, which can reduce the energy consumption required for the separation of solvents.

In the two methods for synthesizing 1,2-pentanediol described above, the main raw material in the first method, a C5 fraction produced in the petrochemical industry, is generally used as a fuel, while there is almost no 1-pentene and 1,2-epoxypentane used to synthesize 1,2-pentanediol. In addition, raw materials have high cost and production technology is backward, which are also important reasons restricting the development of 1,2-pentanediol in China. The second method has the disadvantages of expensive precious metal catalysts, low raw material conversion rate, poor product selectivity, and difficulty in product purification due to more side reactions. Therefore, it is a key to expanding the current production scale of 1,2-pentanediol by utilizing raw materials with wide sources and low costs and developing a synthetic process route with high yield and mild reaction conditions.

### SUMMARY

In view of this, an object of the present disclosure is to provide a method for preparing 1,2-pentanediol. In the present disclosure, the method has low raw material cost, low catalyst cost, high raw material conversion rate, desirable product selectivity, and few side reactions.

To achieve the above object, the present disclosure provides the following technical solutions:

The present disclosure provides a method for preparing 1,2-pentanediol, including:
subjecting 2-hydroxypentanal and hydrogen to hydrogenation reduction under an action of a catalyst to obtain the 1,2-pentanediol; wherein the catalyst is a supported nickel-based catalyst;
the supported nickel-based catalyst includes a carrier and an active component supported on the carrier;
the active component includes a first component and a second component;
the first component is a nickel compound; and
the second component is one or more selected from the group consisting of a copper compound, a cobalt compound, a platinum compound, an iridium compound, a rhodium compound, and a rhenium compound.

In some embodiments, a molar ratio of the 2-hydroxypentanal to the hydrogen is in a range of 1: (50-300).

In some embodiments, the carrier is one or more selected from the group consisting of Al₂O₃, ZnO, Nb₂O₅, SiO₂, TiO₂, CeO₂, and a hydrotalcite (HT) carrier.

In some embodiments, in the catalyst, the first component has a mass percentage of 3% to 20%, and the second component has a mass percentage of 1% to 10%.

In some embodiments, a mass ratio of the 2-hydroxypentanal to the catalyst is in a range of (100-800): 1.

In some embodiments, the hydrogenation reduction is conducted at a temperature of 100° C. to 190° C. and a pressure of 0.5 MPa to 5.0 MPa.

In some embodiments, the method further includes: after the hydrogenation reduction, subjecting a resulting hydrogenation reduction system to steam distillation, adsorption purification, and rectification in sequence.

In some embodiments, the steam distillation is conducted at a steam temperature of 110° C. to 120° C.

In some embodiments, an adsorbent for the adsorption purification includes one or more selected from the group consisting of activated carbon and diatomaceous earth.

In some embodiments, the rectification is conducted at a pressure of 8 mmHg to 20 mmHg and a temperature of 130° C. to 180° C.

The present disclosure provides a method for preparing 1,2-pentanediol, including the following steps: subjecting 2-hydroxypentanal and hydrogen to hydrogenation reduction under an action of a catalyst to obtain the 1,2-pentanediol; wherein the catalyst is a supported nickel-based catalyst; the supported nickel-based catalyst includes a carrier and an active component supported on the carrier; the active component includes a first component and a second component; the first component is a nickel compound; and the second component is one or more selected from the group consisting of a copper compound, a cobalt compound, a platinum compound, an iridium compound, a rhodium compound, and a rhenium compound.

The method provided in the present disclosure has the following advantages:

First, in the method provided by the present disclosure, the 1,2-pentanediol can be prepared by using the 2-hydroxypentanal as a raw material through a one-step hydrogenation reduction process. Compared with traditional processes in which pentene is used as a raw material and subjected to oxidation and hydrolysis, the method has few operating steps, a simple process, and high production efficiency.

Second, in the method provided by the present disclosure, a supported nickel-based catalyst is used, and the second component added to the supported nickel-based catalyst can change the adsorption of carbonyl groups on the surface of the metal active site, thereby significantly improving the selective hydrogenation of aldehyde functional groups. Further, the unreduced second component has a strong interaction with the first component and the carrier, thereby improving both the dispersion and loading stability of the first component. Compared with other types of hydrogenation catalysts, this supported nickel-based catalyst can achieve a feedstock conversion rate of 99% and a product selectivity of 98%, thereby reducing production costs and improving product quality.

Furthermore, the hydrogenation reduction is conducted at a temperature of 100° C. to 190° C. and a pressure of 0.5 MPa to 5.0 MPa. Compared with traditional hydrogenation processes, which are generally conducted at high temperatures under high pressures, the method of the present disclosure has low equipment requirements and high safety.

Furthermore, the method further includes the following steps: after the hydrogenation reduction, subjecting a resulting hydrogenation reduction system to steam distillation, adsorption purification, and rectification in sequence. The purified 1,2-pentanediol reaches a cosmetic grade quality, with no or low odor, and has a product purity of greater than or equal to 99.5%.

### BRIEF DESCRIPTION OF THE DRAWINGS

FIGURE is a schematic diagram showing the mechanism of the hydrogenation reduction.

### DETAILED DESCRIPTION OF THE EMBODIMENTS

The present disclosure provides a method for preparing 1,2-pentanediol, including the following steps:
subjecting 2-hydroxypentanal and hydrogen to hydrogenation reduction under an action of a catalyst to obtain the 1,2-pentanediol; wherein
the catalyst is a supported nickel-based catalyst;
the supported nickel-based catalyst includes a carrier and an active component supported on the carrier;
the active component includes a first component and a second component;
the first component is a nickel compound; and
the second component is one or more selected from the group consisting of a copper compound, a cobalt compound, a platinum compound, an iridium compound, a rhodium compound, and a rhenium compound.

In some embodiments of the present disclosure, the raw materials provided herein are all commercially available products unless otherwise specified.

In some embodiments of the present disclosure, the 2-hydroxypentanal has a purity of 98.5% to 99.5%. In the present disclosure, the 2-hydroxypentanal has a CAS number of 87503-46-6. In the present disclosure, the catalyst is a supported nickel-based catalyst; and the supported nickel-based catalyst includes a carrier and an active component supported on the carrier. In some embodiments of the present disclosure, the carrier is one or more selected from the group consisting of Al₂O₃, ZnO, Nb₂O₅, SiO₂, TiO₂, CeO₂, and an HT carrier. In the present disclosure, the active component includes a first component and a second component; the first component is a nickel compound; and the second component is one or more selected from the group consisting of a copper compound, a cobalt compound, a platinum compound, an iridium compound, a rhodium compound, and a rhenium compound, preferably rhenium compound. In some embodiments of the present disclosure, in the catalyst, the first component has a mass percentage of 3% to 20%, and preferably 8% to 15%, and the second component has a mass percentage of 1% to 10%, and preferably 3% to 7%.

In some embodiments of the present disclosure, the supported nickel-based catalyst is prepared by isovolumetric impregnation, and the isovolumetric impregnation is conducted by a process including the following steps:
subjecting a carrier to first calcination to obtain a calcined carrier;
dissolving a first component salt and a second component salt to obtain a multi-metal salt impregnation liquid; and
impregnating the calcined carrier in the multi-metal salt impregnation liquid, and subjecting a resulting impregnated calcined carrier to drying and second calcination in sequence to obtain the supported nickel-based catalyst.

In the present disclosure, a carrier is subjected to first calcination to obtain a calcined carrier. In some embodiments of the present disclosure, the first calcination is conducted at 500° C., and the first calcination is conducted for 3 h. In the present disclosure, the first calcination is able to remove organic matter and moisture in the carrier.

In the present disclosure, a first component salt and a second component salt are dissolved to obtain a multi-metal salt impregnation liquid. In some embodiments of the present disclosure, the first component salt is nickel nitrate, and preferably nickel nitrate hexahydrate. In some embodiments of the present disclosure, an agent for the dissolving is water. There is no specific limitation on the usage ratio of the first component salt to the second component salt and the usage ratio of the first component salt to the carrier, which can be set according to the catalyst to be prepared.

In the present disclosure, the calcined carrier is impregnated in the multi-metal salt impregnation liquid, and a resulting impregnated calcined carrier is subjected to drying and second calcination in sequence to obtain the supported nickel-based catalyst.

In some embodiments of the present disclosure, the impregnation is conducted at room temperature, and the impregnation is conducted for 24 h. In some embodiments of the present disclosure, the drying is conducted at 110° C. In some embodiments of the present disclosure, the second calcination is conducted at 500° C.; heating to the temperature of the second calcination is conducted at a rate of 10° C./min; the second calcination is conducted for 3 h; and the second calcination is conducted in an air atmosphere. In some embodiments of the present disclosure, the second calcination is conducted in a muffle furnace. In the present disclosure, the second calcination is able to decompose the metal salt to obtain a black catalyst.

In some embodiments of the present disclosure, the catalyst is subjected to activation before use; the activation is conducted in an H₂—Ar mixed gas; a volume fraction of H₂ in the H₂—Ar mixed gas is 5%; the activation is conducted at 500° C.; heating to the temperature of the activation is conducted at a rate of 10° C./min; the activation is conducted for 3 h; and the activation is conducted in a fixed bed reactor. In the present disclosure, the activation is able to reduce the oxidized nickel-based catalyst into the reduced nickel-based catalyst, thereby making the catalyst active.

In some embodiments of the present disclosure, a molar ratio of the 2-hydroxypentanal to the hydrogen is in a range of 1: (50-300), and preferably 1: (60-200). In some embodiments of the present disclosure, the molar ratio of the 2-hydroxypentanal to the hydrogen is in a range of 1: (50-300), wherein the amount of the hydrogen refers to the amount introduced and does not refer to the amount actually participating in the reaction. In some embodiments of the present disclosure, a mass ratio of the 2-hydroxypentanal to the catalyst is in a range of (100-800): 1, and preferably (200-400): 1.

In some embodiments of the present disclosure, the hydrogenation reduction is conducted at a temperature of 100° C. to 190° C., and the hydrogenation reduction is conducted at a pressure of 0.5 MPa to 5.0 MPa.

In some embodiments of the present disclosure, the hydrogenation reduction is conducted in a hydrogenation tower. In conjunction with the hydrogenation tower, an operation of the hydrogenation reduction of the 2-hydroxypentanal and hydrogen under the action of the catalyst is described below, which is conducted by a process including: gasifying the 2-hydroxypentanal to obtain gaseous 2-hydroxypentanal, wherein the catalyst is fixed in a middle part of the hydrogenation tower; and introducing the gaseous 2-hydroxypentanal into the hydrogenation tower through a top part, introducing the hydrogen into the hydrogenation tower through the top part, and contacting the gaseous 2-hydroxypentanal and hydrogen at the catalyst site and conducting hydrogenation reaction. There is no specific limitation on the gasification of the 2-hydroxypentanal, as long as the gaseous 2-hydroxypentanal could be obtained. There is no specific limitation on the flow rates of the gaseous 2-hydroxypentanal and hydrogen, as long as the two components could satisfy the usage ratio and be introduced simultaneously.

In the present disclosure, the schematic diagram showing the mechanism of the hydrogenation reduction is shown in the FIGURE. As shown in the FIGURE, hydrogen is dissociated and adsorbed on the surface of the active component of the catalyst, namely bimetallic nanoparticles, to obtain activated hydrogen; meanwhile, due to the geometric configuration and electronic properties of the bimetallic nanoparticles, C═O functional group in the 2-hydroxypentanal could be selectively adsorbed on the surface of the bimetallic nanoparticles. Then activated high-energy hydrogen atoms are transferred to oxygen atom and carbon atom of the C═O functional group, such that the aldehyde group is reduced to hydroxymethyl. Due to the poor adsorption capacity of the hydroxyl group on the surface of bimetallic nanoparticles, the obtained 1,2-pentanediol could be quickly desorbed from the catalyst surface, thereby completing the whole catalytic hydrogenation. During the whole process of catalytic hydrogenation, due to the synergistic effect between the bimetals, the adsorption of the hydroxyl group on the metal surface is poor, as a result, the side reaction of nickel on the hydrogenation and deoxygenation of the substrate could be largely avoided, thereby significantly improving the selectivity of the target product.

In the present disclosure, the hydrogenation reduction is conducted according to a main reaction formula below:

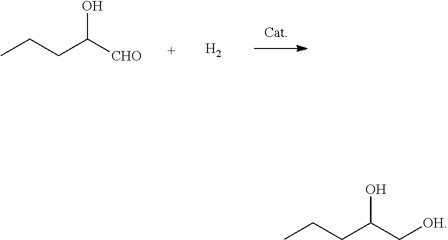

The inevitable side reactions are as follows:

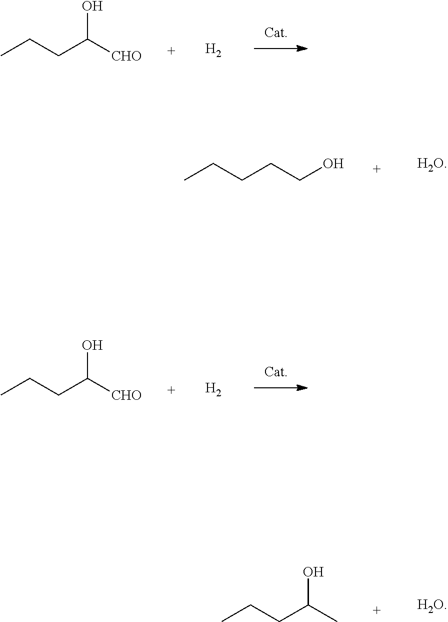

In some embodiments of the present disclosure, the method further includes the following steps: after the hydrogenation reduction, subjecting a resulting hydrogenation reduction system to steam distillation, adsorption purification, and rectification in sequence. In some embodiments of the present disclosure, the steam distillation is conducted at a steam temperature of 110° C. to 120° C., and the steam distillation is conducted in an atmospheric distillation tower. In some embodiments of the present disclosure, an adsorbent for the adsorption purification includes one or more selected from the group consisting of activated carbon and diatomaceous earth. In some embodiments of the present disclosure, the rectification is conducted at a pressure of 8 mmHg to 20 mmHg, and preferably 10 mmHg to 15 mmHg; and the rectification is conducted at a temperature of 130° C. to 180° C., and preferably 150° C. to 160° C.

The method for preparing 1,2-pentanediol provided by the present disclosure is described in detail below with reference to the examples, but these examples may not be understood as a limitation to the scope of the present disclosure.

### EXAMPLES

In Examples 1 to 10, hydrogenation reduction was conducted according to the material ratios and conditions as shown in Table 1, and the results of the hydrogenation reduction are listed in Table 1.

TABLE 1
Reaction conditions, product purity, and product batch yield of Examples 1 to 10

| | Item | | | | | | |
|---|---|---|---|---|---|---|---|
| | | | | | Hydrogenation | | |
| | 2- | | Hydrogen | Hydrogenation | reduction | | 1,2- |
| | Hydroxypentanal | Catalyst | consumption/ | reduction | temperature/ | Product | Pentanediol |
| Item | dosage/kg | dosage/kg | kg | pressure/MPa | ° C. | purity | yield/kg |
| Example 1 | 500 | 2 | 9.98 | 0.5 | 190 | 99.50% | 397 |
| Example 2 | 500 | 2 | 10.09 | 1.0 | 180 | 99.56% | 402 |
| Example 3 | 500 | 2 | 10.16 | 1.5 | 170 | 99.51% | 408 |
| Example 4 | 500 | 2 | 10.25 | 2.0 | 160 | 99.63% | 406 |
| Example 5 | 500 | 2 | 10.32 | 2.5 | 150 | 99.60% | 403 |
| Example 6 | 500 | 2 | 10.24 | 3.0 | 140 | 99.58% | 400 |
| Example 7 | 500 | 2 | 10.19 | 3.5 | 130 | 99.55% | 407 |
| Example 8 | 500 | 2 | 10.10 | 4.0 | 120 | 99.53% | 398 |
| Example 9 | 500 | 2 | 10.05 | 4.5 | 110 | 99.55% | 395 |
| Example 10 | 500 | 2 | 10.01 | 5.0 | 100 | 99.52% | 390 |

In Examples 1 to 10, the supported nickel-based catalyst was conducted by a process as follows:
(1) A carrier Al₂O₃ was calcined at 500° C. for 3 h to remove impurities, such as organic matter, and water in the carrier to obtain a calcined carrier.
(2) 1,067.78 g of Ni(NO₃)₂·6H₂O and 186.29 g of a rhenium metal compound NH₄ReO₄ were dissolved in 1,435.71 g of deionized water to obtain a bimetallic impregnation liquid with a nickel salt concentration of 2.0 mol/L and a rhenium metal compound concentration of 0.38 mol/L.
(3) 2,155.17 g of the calcined carrier was added into the bimetallic impregnation liquid under vigorous stirring, impregnated at room temperature for 24 h, and then dried at 110° C. overnight to obtain a green powder.
(4) The green powder was placed in a muffle furnace, heated to 500° C. at 10° C./min in an air atmosphere, and then calcined for 3 h to decompose the metal salt to obtain a black supported nickel-based catalyst.

### Activation of the Supported Nickel-Based Catalyst

The black supported nickel-based catalyst was placed in a fixed bed reactor, 5% H₂/Ar gas was introduced into the fixed bed reactor, and the catalyst was heated to 500° C. at 10° C./min and subjected to activation for 3 h at 500° C. to obtain an activated supported nickel-based catalyst.

In Examples 1 to 10, a total amount of hydrogen introduced was 1,000 kg, and a post-treatment was conducted by a process as follows:

A resulting hydrogenation reduction system was subjected to steam distillation in an atmospheric distillation tower, at a steam temperature of 115° C., an activated carbon was added thereto and subjected to adsorption purification, and then an adsorbed system was subjected to rectification at 15 mmHg and 155° C.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. A method for preparing 1,2-pentanediol, comprising:
subjecting 2-hydroxypentanal and hydrogen to hydrogenation reduction under an action of a catalyst to obtain the 1,2-pentanediol; wherein
the catalyst is a supported nickel-based catalyst;
the supported nickel-based catalyst comprises a carrier and an active component supported on the carrier;
the active component comprises a first component and a second component;
the first component is a nickel compound; and
the second component is one or more selected from the group consisting of a copper compound, a cobalt compound, a platinum compound, an iridium compound, a rhodium compound, and a rhenium compound.

2. The method of claim 1, wherein a molar ratio of the 2-hydroxypentanal to the hydrogen is in a range of 1:50-300.

3. The method of claim 1, wherein the carrier is one or more selected from the group consisting of Al₂O₃, ZnO, Nb₂O₅, SiO₂, TiO₂, CeO₂, and a hydrotalcite (HT) carrier.

4. The method of claim 1, wherein in the catalyst, the first component has a mass percentage of 3% to 20%, and the second component has a mass percentage of 1% to 10%.

5. The method of claim 1, wherein a mass ratio of the 2-hydroxypentanal to the catalyst is in a range of 100-800:1.

6. The method of claim 1, wherein the hydrogenation reduction is conducted at a temperature of 100° C. to 190° C. and a pressure of 0.5 MPa to 5.0 MPa.

7. The method of claim 1, further comprising: after the hydrogenation reduction, subjecting a resulting hydrogenation reduction system to steam distillation, adsorption purification, and rectification in sequence.

8. The method of claim 7, wherein the steam distillation is conducted at a steam temperature of 110° C. to 120° C.

9. The method of claim 7, wherein an adsorbent for the adsorption purification is one or more selected from the group consisting of activated carbon and diatomaceous earth.

10. The method of claim 7, wherein the rectification is conducted at a pressure of 8 mmHg to 20 mmHg and a temperature of 130° C. to 180° C.

11. The method of claim 3, wherein a mass ratio of the 2-hydroxypentanal to the catalyst is in a range of 100-800:1.

12. The method of claim 4, wherein a mass ratio of the 2-hydroxypentanal to the catalyst is in a range of 100-800:1.

13. The method of claim 6, further comprising: after the hydrogenation reduction, subjecting a resulting hydrogenation reduction system to steam distillation, adsorption purification, and rectification in sequence.
